(19) United States
(12) Patent Application Publication
(10) Pub. No.: US 2023/0206447 A1
(43) Pub. Date: Jun. 29, 2023

(54) IMAGE ENCODING DEVICE, IMAGE ENCODING METHOD, IMAGE ENCODING PROGRAM, IMAGE DECODING DEVICE, IMAGE DECODING METHOD, IMAGE DECODING PROGRAM, IMAGE PROCESSING DEVICE, LEARNING DEVICE, LEARNING METHOD, LEARNING PROGRAM, SIMILAR IMAGE SEARCH DEVICE, SIMILAR IMAGE SEARCH METHOD, AND SIMILAR IMAGE SEARCH PROGRAM

(71) Applicants: NATIONAL CANCER CENTER, Tokyo (JP); FUJIFILM Corporation, Tokyo (JP)

(72) Inventors: Kazuma KOBAYASHI, Tokyo (JP); Mototaka MIYAKE, Tokyo (JP); Ryuji HAMAMOTO, Tokyo (JP); Jun MASUMOTO, Tokyo (JP)

(73) Assignees: NATIONAL CANCER CENTER, Tokyo (JP); FUJIFILM Corporation, Tokyo (JP)

(21) Appl. No.: 18/177,733

(22) Filed: Mar. 2, 2023

Related U.S. Application Data
(63) Continuation of application No. PCT/JP2021/026147, filed on Jul. 12, 2021.

(30) Foreign Application Priority Data
Sep. 15, 2020 (JP) 2020-154532

(51) Int. Cl. G06T 7/00; G06V 10/25; G06V 10/774; G06V 20/70; G06V 10/74; G06V 10/77; G06F 16/532

(52) U.S. Cl. CPC G06T 7/0014; G06V 10/25; G06V 10/774; G06V 20/70; G06V 10/761; G06V 10/7715; G06F 16/532; G06V 2201/03; G06T 2200/04; G06T 2207/30016; G06T 2207/20081; G06T 2207/20076; G06T 2207/20021

(57) ABSTRACT

A processor encodes a target image to derive at least one first feature amount indicating an image feature for an abnormality of a region of interest included in the target image. In addition, the processor encodes the target image to derive at least one second feature amount indicating an image feature for an image in a case in which the region of interest included in the target image is a normal region.

### CROSS-REFERENCE TO RELATED APPLICATIONS

The present application is a Continuation of PCT International Application No. PCT/JP2021/026147, filed on Jul. 12, 2021, which claims priority to Japanese Patent Application No. 2020-154532, filed on Sep. 15, 2020. Each application above is hereby expressly incorporated by reference, in its entirety, into the present application.

### BACKGROUND

### Technical Field

The present disclosure relates to an image encoding device, an image encoding method, an image encoding program, an image decoding device, an image decoding method, an image decoding program, an image processing device, a learning device, a learning method, a learning program, a similar image search device, a similar image search method, and a similar image search program.

### Related Art

In recent years, various methods for detecting a region of interest from medical images acquired by medical apparatuses, such as a computed tomography (CT) apparatus and a magnetic resonance imaging (MRI) apparatus, have been proposed. For example, JP2020-062355A discloses a method that extracts a lesion region from a medical image as an extraction target, using a learning model that has extracted first data related to an image of a first region, which is a region inside a lesion, second data related to an image of a second region, which is a region around the lesion, and third data related to an image of a third region, which is a region outside the lesion, from medical image data for training and that has learned the extracted data. The learning model disclosed in JP2020-062355A extracts the lesion region from the target medical image, using a feature amount of the lesion region and a feature amount of the region around the lesion.

Meanwhile, it is possible to efficiently perform a diagnosis with reference to a past medical image that is similar to a case for the region of interest included in the medical image. Therefore, a method has been proposed which searches for a past medical image that is similar to a target medical image (for example, see JP2004-05364A). The method disclosed in JP2004-05364A first derives a feature amount of a region of interest included in a medical image to be diagnosed. Then, the method derives a similarity on the basis of a difference between a feature amount derived in advance for a medical image stored in a database and a feature amount derived from the target medical image and searches for a similar past medical image on the basis of the similarity.

However, it can be said that an image feature of a region of interest, such as a lesion, in a medical image is a combination of a pathological change caused by a disease and normal anatomical features which are originally present therein. The normal anatomical features of the human body are common. Therefore, with a focus on the region of interest, a clinician extracts the normal anatomical features, which are present behind the region of interest, and evaluates the region of interest, assuming an image feature that purely reflects abnormality.

Therefore, it is very important in image diagnosis to compare and interpret disease regions in medical images of the same patient before and after a disease occurs and to compare and interpret medical images of different patients having a similar lesion. In order to reproduce the process, in which the clinician recognizes the medical images, with a computer, it is necessary to express the image feature of the region of interest as a difference from the normal anatomical features that are originally present therein. In addition, at the same time, it is also necessary to reproduce the normal anatomical features in a case in which the region of interest is a normal region.

However, the method disclosed in JP2020-062355A only detects the region of interest from the medical image. In addition, the method disclosed in JP2004-05364A only searches for the medical images having a similar region of interest in the images. Therefore, even in a case in which the methods disclosed in JP2020-062355A and JP2004-05364A are used, it is difficult to separately treat the image feature of the region of interest included in the medical image and the image feature in a case in which the region of interest is a normal region.

### SUMMARY OF THE INVENTION

The present disclosure has been made in view of the above circumstances, and an object of the present disclosure is to provide a technique that can separately treat an image feature for an abnormality of a region of interest and an image feature for an image in a case in which the region of interest is a normal region for a target image that includes an abnormal region as the region of interest.

An image encoding device according to the present disclosure comprises at least one processor. The processor is configured to encode a target image to derive at least one first feature amount indicating an image feature for an abnormality of a region of interest included in the target image and to encode the target image to derive at least one second feature amount indicating an image feature for an image in a case in which the region of interest included in the target image is a normal region.

In addition, the image encoding device according to the present disclosure may extract the region of interest while deriving at least one of the first feature amount or the second feature amount. Alternatively, the region of interest may have already been extracted from the target image. Further, the region of interest may be extracted from the target image in response to the input of an operator on the displayed target image.

In the present disclosure, the image feature for the “abnormality” of the region of interest may be expressed as a difference between image features indicating how much the image feature for the region of interest included in the actual target image deviates from the image feature for the image in a case in which the region of interest in the target image is a normal region.

In addition, in the image encoding device according to the present disclosure, a combination of the first feature amount and the second feature amount may indicate an image feature for the target image.

Further, the image encoding device according to the present disclosure may further comprise a storage that stores at least one first feature vector indicating a representative image feature for the abnormality of the region of interest and at least one second feature vector indicating a representative image feature for the image in a case in which the region of interest is the normal region. The processor may be configured to derive the first feature amount by substituting a feature vector indicating the image feature for the abnormality of the region of interest with a first feature vector, which minimizes a difference from the image feature for the abnormality of the region of interest, among the first feature vectors to quantize the feature vector, and to derive the second feature amount by substituting a feature vector indicating the image feature for the image in a case in which the region of interest is the normal region with a second feature vector, which minimizes a difference from the image feature for the image in a case in which the region of interest is the normal region, among the second feature vectors to quantize the feature vector.

Furthermore, in the image encoding device according to the present disclosure, the processor may be configured to derive the first feature amount and the second feature amount, using an encoding learning model which has been trained to derive the first feature amount and the second feature amount in a case in which the target image is input.

An image decoding device according to the present disclosure comprises at least one processor. The processor is configured to extract a region corresponding to a type of the abnormality of the region of interest in the target image on the basis of the first feature amount derived from the target image by the image encoding device according to the present disclosure.

In addition, in the image decoding device according to the present disclosure, the processor may be configured to derive a first reconstructed image obtained by reconstructing an image feature for an image in a case in which the region of interest in the target image is a normal region on the basis of the second feature amount and to derive a second reconstructed image obtained by reconstructing an image feature for the target image on the basis of the first feature amount and the second feature amount.

Further, in the image decoding device according to the present disclosure, the processor may be configured to derive a label image corresponding to the type of the abnormality of the region of interest in the target image, the first reconstructed image, and the second reconstructed image, using a decoding learning model which has been trained to derive the label image corresponding to the type of the abnormality of the region of interest in the target image on the basis of the first feature amount, to derive the first reconstructed image obtained by reconstructing the image feature for the image in a case in which the region of interest in the target image is the normal region on the basis of the second feature amount, and to derive the second reconstructed image obtained by reconstructing the image feature of the target image on the basis of the first feature amount and the second feature amount.

An image processing device according to the present disclosure comprises the image encoding device according to the present disclosure and the image decoding device according to the present disclosure.

According to the present disclosure, there is provided a learning device that trains the encoding learning model in the image encoding device according to the present disclosure and the decoding learning model in the image decoding device according to the present disclosure, using training data consisting of a training image including a region of interest and a training label image corresponding to a type of an abnormality of the region of interest in the training image. The learning device comprises at least one processor. The processor is configured to derive a first learning feature amount and a second learning feature amount corresponding to the first feature amount and the second feature amount, respectively, from the training image using the encoding learning model, to derive a learning label image corresponding to the type of the abnormality of the region of interest included in the training image on the basis of the first learning feature amount, to derive a first learning reconstructed image obtained by reconstructing an image feature for an image in a case in which the region of interest in the training image is a normal region on the basis of the second learning feature amount, and to derive a second learning reconstructed image obtained by reconstructing an image feature for the training image on the basis of the first learning feature amount and the second learning feature amount, using the decoding learning model, and to train the encoding learning model and the decoding learning model such that at least one of a first loss which is a difference between the first learning feature amount and a predetermined probability distribution of the first feature amount, a second loss which is a difference between the second learning feature amount and a predetermined probability distribution of the second feature amount, a third loss based on a difference between the training label image included in the training data and the learning label image as semantic segmentation for the training image, a fourth loss based on a difference between the first learning reconstructed image and an image outside the region of interest in the training image, a fifth loss based on a difference between the second learning reconstructed image and the training image, or a sixth loss based on a difference between regions corresponding to an inside and an outside of the region of interest in the first learning reconstructed image and in the second learning reconstructed image satisfies a predetermined condition.

The “difference as semantic segmentation” for the third loss is an index determined on the basis of the overlap between a region corresponding to the type of the abnormality indicated by the training label image and a region corresponding to the type of the abnormality indicated by the learning label image.

The “outside of the region of interest” for the fourth loss means all regions other than the region of interest in the training image. In addition, in a case in which the training image includes a background that does not include any structure, the outside of the region of interest also includes a region including the background. On the other hand, the outside of the region of interest may include only a region that does not include the background.

The “regions corresponding to the inside and outside of the region of interest” for the sixth loss mean both regions which correspond to the region of interest and regions which do not correspond to the region of interest in the first learning reconstructed image and in the second learning reconstructed image. The region that does not correspond to the region of interest means all regions other than the region corresponding to the region of interest in the first learning reconstructed image and in the second learning reconstructed image. In addition, in a case in which the first and second learning reconstructed images include a background that does not include any structure, the region that does not correspond to the region of interest also includes a region including the background. On the other hand, the region that does not correspond to the region of interest may include only a region that does not include the background.

A similar image search device according to the present disclosure comprises: at least one processor; and the image encoding device according to the present disclosure. The processor is configured to derive a first feature amount and a second feature amount for a query image using the image encoding device, to derive a similarity between the query image and each of a plurality of reference images on the basis of at least one of the first feature amount or the second feature amount derived from the query image with reference to an image database in which a first feature amount and a second feature amount for each of the plurality of reference images are registered in association with each of the plurality of reference images, and to extract a reference image that is similar to the query image as a similar image from the image database on the basis of the similarity.

An image encoding method according to the present disclosure comprises: encoding a target image to derive at least one first feature amount indicating an image feature for an abnormality of a region of interest included in the target image; and encoding the target image to derive at least one second feature amount indicating an image feature for an image in a case in which the region of interest included in the target image is a normal region.

An image decoding method according to the present disclosure comprises extracting a region corresponding to a type of an abnormality of the region of interest in the target image on the basis of the first feature amount derived from the target image by the image encoding device according to the present disclosure.

According to the present disclosure, there is provided a learning method for training the encoding learning model in the image encoding device according to the present disclosure and the decoding learning model in the image decoding device according to the present disclosure, using training data consisting of a training image including a region of interest and a training label image corresponding to a type of an abnormality of the region of interest in the training image. The learning method comprises: deriving a first learning feature amount and a second learning feature amount corresponding to the first feature amount and the second feature amount, respectively, from the training image using the encoding learning model; deriving a learning label image corresponding to the type of the abnormality of the region of interest included in the training image on the basis of the first learning feature amount, deriving a first learning reconstructed image obtained by reconstructing an image feature for an image in a case in which the region of interest in the training image is a normal region on the basis of the second learning feature amount, and deriving a second learning reconstructed image obtained by reconstructing an image feature for the training image on the basis of the first learning feature amount and the second learning feature amount, using the decoding learning model; and training the encoding learning model and the decoding learning model such that at least one of a first loss which is a difference between the first learning feature amount and a predetermined probability distribution of the first feature amount, a second loss which is a difference between the second learning feature amount and a predetermined probability distribution of the second feature amount, a third loss based on a difference between the training label image included in the training data and the learning label image as semantic segmentation for the training image, a fourth loss based on a difference between the first learning reconstructed image and an image outside the region of interest in the training image, a fifth loss based on a difference between the second learning reconstructed image and the training image, or a sixth loss based on a difference between regions corresponding to an inside and an outside of the region of interest in the first learning reconstructed image and in the second learning reconstructed image satisfies a predetermined condition.

A similar image search method according to the present disclosure comprises: deriving a first feature amount and a second feature amount for a query image using the image encoding device according to the present disclosure; deriving a similarity between the query image and each of a plurality of reference images on the basis of at least one of the first feature amount or the second feature amount derived from the query image with reference to an image database in which a first feature amount and a second feature amount for each of the plurality of reference images are registered in association with each of the plurality of reference images; and extracting a reference image that is similar to the query image as a similar image from the image database on the basis of the similarity.

In addition, programs that cause a computer to execute the image encoding method, the image decoding method, the learning method, and the similar image search method according to the present disclosure may be provided.

According to the present disclosure, it is possible to separately treat an image feature for an abnormality of a region of interest and an image feature for an image in a case in which the region of interest is a normal region for a target image that includes an abnormal region as the region of interest.

### BRIEF DESCRIPTION OF THE DRAWINGS

FIG. 1 is a diagram illustrating a schematic configuration of a medical information system to which an image encoding device, an image decoding device, a learning device, and a similar image search device according to an embodiment of the present disclosure are applied.

FIG. 2 is a diagram illustrating a schematic configuration of an image processing system according to this embodiment.

FIG. 3 is a functional configuration diagram illustrating the image processing system according to this embodiment.

FIG. 4 is a conceptual diagram illustrating processes performed by the image encoding device and the image decoding device according to this embodiment.

FIG. 5 is a diagram illustrating substitution with a first feature vector.

FIG. 6 is a diagram illustrating an example of training data used for learning.

FIG. 7 is a diagram illustrating a search result list.

FIG. 8 is a diagram illustrating a display screen for a search result according to a first search condition.

FIG. 9 is a diagram illustrating a display screen for a search result according to a second search condition.

FIG. 10 is a diagram illustrating a display screen for a search result according to a third search condition.

FIG. 11 is a flowchart illustrating a learning process performed in this embodiment.

FIG. 12 is a flowchart illustrating a similar image search process performed in this embodiment.

### DETAILED DESCRIPTION

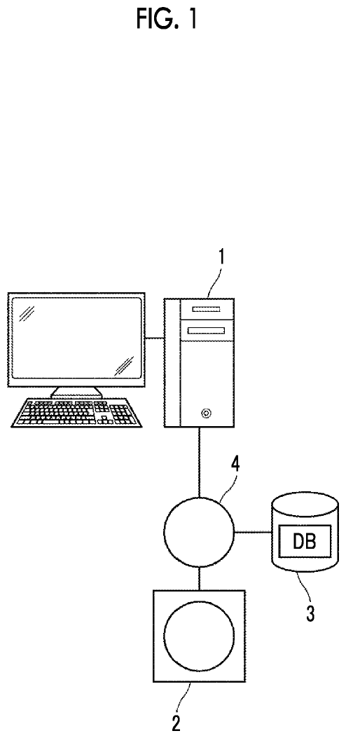

Hereinafter, an embodiment of the present disclosure will be described with reference to the drawings. First, a configuration of a medical information system to which an image encoding device, an image decoding device, a learning device, and a similar image search device according to this embodiment are applied will be described. In addition, in the following description, an image processing device includes the image encoding device and the image decoding device according to the present disclosure. FIG. 1 is a diagram illustrating a schematic configuration of the medical information system. In the medical information system illustrated in FIG. 1, a computer 1 including the image processing device, the learning device, and the similar image search device according to this embodiment, an imaging apparatus 2, and an image storage server 3 are connected through a network 4 such that they can communicate with each other.

The computer 1 includes the image processing device, the learning device, and the similar image search device according to this embodiment, and an image encoding program, an image decoding program, a learning program, and a similar image search program according to this embodiment are installed in the computer 1. The computer 1 may be a workstation or a personal computer that is directly operated by a doctor who performs diagnosis or may be a server computer that is connected to them through the network. In addition, the image encoding program, the image decoding program, the learning program, and the similar image search program are stored in a storage device of a server computer connected to the network or in a network storage to be accessed from the outside and are downloaded and installed in the computer 1 used upon request by the doctor. Alternatively, the programs are recorded on a recording medium, such as a digital versatile disc (DVD) or a compact disc read only memory (CD-ROM), are distributed, and are installed in the computer 1 from the recording medium.

The imaging apparatus 2 is an apparatus that images a diagnosis target part of a subject and that generates a three-dimensional image indicating the part and is specifically a computed tomography (CT) apparatus, a magnetic resonance imaging (MM) apparatus, a positron emission tomography (PET) apparatus, or the like. The three-dimensional image, which has been generated by the imaging apparatus 2 and consists of a plurality of slice images, is transmitted to the image storage server 3 and is then stored therein. In addition, in this embodiment, a diagnosis target part of a patient that is the subject is a brain, and the imaging apparatus 2 is an MRI apparatus and generates an MRI image of a head including the brain of the subject as the three-dimensional image.

The image storage server 3 is a computer that stores and manages various types of data and that comprises a high-capacity external storage device and database management software. The image storage server 3 performs communication with other apparatuses through the wired or wireless network 4 to transmit and receive, for example, image data. Specifically, the image storage server 3 acquires various types of data including the image data of the three-dimensional image generated by the imaging apparatus 2 through the network, stores the acquired data in a recording medium, such as a high-capacity external storage device, and manages the data. In addition, the storage format of the image data and the communication between the apparatuses through the network 4 are based on a protocol such as digital imaging and communication in medicine (DICOM). Further, the image storage server 3 stores training data which will be described below.

Further, in this embodiment, an image database DB is stored in the image storage server 3. A plurality of images including various diseases, such as cerebral hemorrhage and cerebral infarction, are registered as reference images in the image database DB. The image database DB will be described below. Further, in this embodiment, the reference image is also a three-dimensional image consisting of a plurality of slice images.

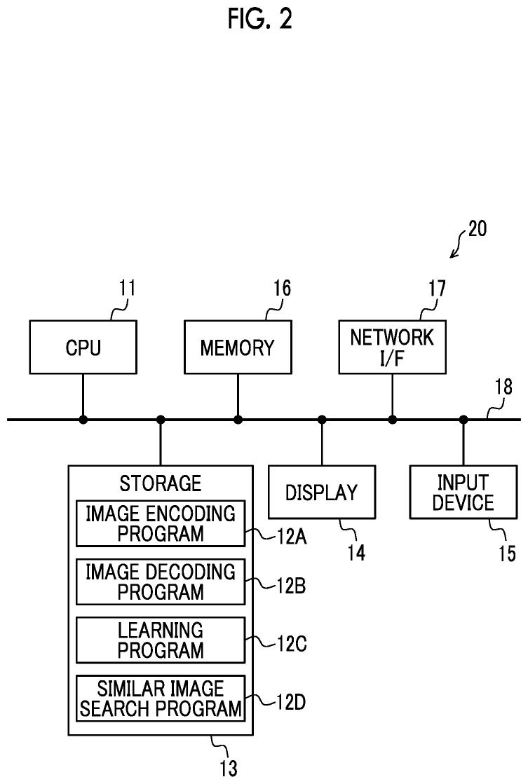

Next, the image encoding device, the image decoding device, the learning device, and the similar image search device according to this embodiment will be described. FIG. 2 illustrates a hardware configuration of an image processing system including the image encoding device, the image decoding device, the learning device, and the similar image search device according to this embodiment. As illustrated in FIG. 2, an image processing system 20 according to this embodiment includes a central processing unit (CPU) 11, a non-volatile storage 13, and a memory 16 as a temporary storage area. In addition, the image processing system 20 includes a display 14, such as a liquid crystal display, an input device 15, such as a keyboard and a mouse, and a network interface (I/F) 17 that is connected to the network 4. The CPU 11, the storage 13, the display 14, the input device 15, the memory 16, and the network I/F 17 are connected to a bus 18. In addition, the CPU 11 is an example of a processor according to the present disclosure.

The storage 13 is implemented by, for example, a hard disk drive (HDD), a solid state drive (SSD), and a flash memory. An image encoding program 12A, an image decoding program 12B, a learning program 12C, and a similar image search program 12D are stored in the storage 13 as a storage medium. The CPU 11 reads the image encoding program 12A, the image decoding program 12B, the learning program 12C, and the similar image search program 12D from the storage 13, develops them in the memory 16, and executes the developed image encoding program 12A, image decoding program 12B, learning program 12C, and similar image search program 12D.

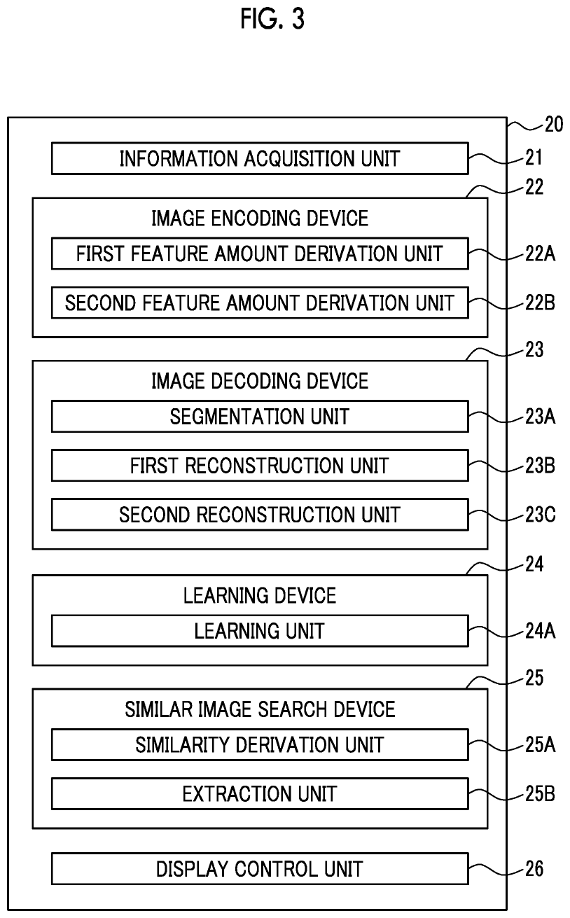

Next, a functional configuration of the image processing system according to this embodiment will be described. FIG. 3 is a diagram illustrating the functional configuration of the image processing system according to this embodiment. As illustrated in FIG. 3, the image processing system 20 according to this embodiment comprises an information acquisition unit 21, an image encoding device 22, an image decoding device 23, a learning device 24, a similar image search device 25, and a display control unit 26. The image encoding device 22 comprises a first feature amount derivation unit 22A and a second feature amount derivation unit 22B. The image decoding device 23 comprises a segmentation unit 23A, a first reconstruction unit 23B, and a second reconstruction unit 23C. The learning device 24 comprises a learning unit 24A. The similar image search device 25 comprises a similarity derivation unit 25A and an extraction unit 25B. In addition, the image encoding device 22 may comprise the information acquisition unit 21. Further, the similar image search device 25 may comprise the display control unit 26.

The CPU 11 executes the image encoding program 12A, the image decoding program 12B, the learning program 12C, and the similar image search program 12D to function as the information acquisition unit 21, the first feature amount derivation unit 22A, the second feature amount derivation unit 22B, the segmentation unit 23A, the first reconstruction unit 23B, the second reconstruction unit 23C, the learning unit 24A, the similarity derivation unit 25A, the extraction unit 25B, and the display control unit 26.

The information acquisition unit 21 acquires a query image to be searched for, which will be described below, as a target image from the image storage server 3 in response to an instruction from an operator through the input device 15. Here, in the following description of the image encoding device 22 and the image decoding device 23, an image that is input to the image encoding device 22 is referred to as the target image. Meanwhile, an image that is input to the image encoding device 22 in a case in which the learning device 24 performs learning is a training image. In addition, in a case in which the similar image search device 25 is described, an image that is input to the image encoding device 22 is referred to as the query image.

Further, in a case in which the target image has already been stored in the storage 13, the information acquisition unit 21 may acquire the target image from the storage 13. In addition, the information acquisition unit 21 acquires a plurality of training data items from the image storage server 3 in order to train an encoding learning model and a decoding learning model which will be described below.

The first feature amount derivation unit 22A constituting the image encoding device 22 encodes the target image to derive at least one first feature amount indicating an image feature for the abnormality of a region of interest included in the target image. Further, in this embodiment, the region of interest is extracted while the first feature amount is being derived. In addition, the region of interest may be extracted in advance from the target image before the first feature amount is derived. For example, the image encoding device 22 may be provided with a function of detecting the region of interest from the target image, and the region of interest may be extracted from the target image before the image encoding device 22 derives the first feature amount. Alternatively, the region of interest may have already been extracted from the target image stored in the image storage server 3. In addition, the target image may be displayed on the display 14, and the region of interest may be extracted from the target image in response to the input of the operator on the displayed target image.

The second feature amount derivation unit 22B constituting the image encoding device 22 encodes the target image to derive at least one second feature amount indicating an image feature for the image in a case in which the region of interest included in the target image is a normal region.

Therefore, the first feature amount derivation unit 22A and the second feature amount derivation unit 22B have an encoder and a latent model as an encoding learning model which has been trained to derive the first feature amount and the second feature amount in a case in which the target image is input. Further, in this embodiment, it is assumed that the first feature amount derivation unit 22A and the second feature amount derivation unit 22B have a common encoding learning model. The encoder and the latent model as the encoding learning model will be described below.

Further, in this embodiment, the target image includes the brain, and the region of interest is a region determined according to the type of brain disease, such as cerebral infarction or cerebral hemorrhage.

Here, the second feature amount indicates an image feature for the image in a case in which the region of interest in the target image is a normal region. Therefore, the second feature amount indicates an image feature obtained by interpolating the region of interest in the target image, that is, a disease region, with an image feature of the region in which a disease is not present, particularly, the normal tissue of the brain. Therefore, the second feature amount indicates the image feature of the image in a state in which all of the tissues of the brain in the target image are normal.

In addition, a combination of the first feature amount and the second feature amount may indicate the image feature of the target image, particularly, the image feature of the brain including the region determined according to the type of disease. In this case, the first feature amount indicates an image feature for the abnormality of the region of interest included in the target image and indicates an image feature representing the difference from the image feature in a case in which the region of interest included in the target image is a normal region. In this embodiment, since the region of interest is a brain disease, the first feature amount indicates an image feature representing the difference from the image feature of the image in a state in which all of the tissues of the brain in the target image are normal. Therefore, it is possible to separately acquire an image feature for the abnormality of the region determined according to the type of disease and an image feature of the image in a state in which all of the tissues of the brain are normal from the image of the brain which includes an abnormal region as the region of interest.

The segmentation unit 23A of the image decoding device 23 derives a region-of-interest label image corresponding to the type of the abnormality of the region of interest in the target image on the basis of the first feature amount derived by the first feature amount derivation unit 22A.

The first reconstruction unit 23B of the image decoding device 23 derives a first reconstructed image obtained by reconstructing the image feature for the image in a case in which the region of interest in the target image is a normal region, on the basis of the second feature amount derived by the second feature amount derivation unit 22B.

The second reconstruction unit 23C of the image decoding device 23 derives a second reconstructed image obtained by reconstructing the image feature of the target image on the basis of the first feature amount derived by the first feature amount derivation unit 22A and the second feature amount derived by the second feature amount derivation unit 22B. In addition, the image feature of the reconstructed target image is an image feature including a background other than the brain included in the target image.

Therefore, the segmentation unit 23A, the first reconstruction unit 23B, and the second reconstruction unit 23C have a decoder as a decoding learning model which has been trained to derive the region-of-interest label image corresponding to the type of the abnormality of the region of interest in a case in which the first feature amount and the second feature amount are input and to derive the first reconstructed image and the second reconstructed image.

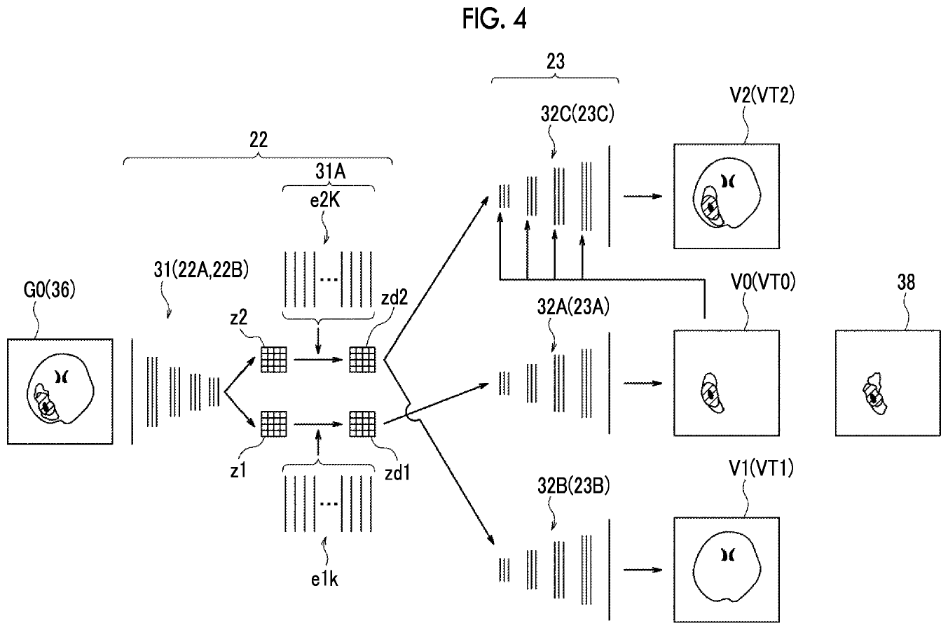

FIG. 4 is a conceptual diagram illustrating a process performed by the image encoding device and the image decoding device according to this embodiment. As illustrated in FIG. 4, the image encoding device 22 includes an encoder 31 and a latent model 31A which are the encoding learning model. The encoder 31 and the latent model 31A have the functions of the first feature amount derivation unit 22A and the second feature amount derivation unit 22B according to this embodiment. In addition, the image decoding device 23 includes decoders 32Ato 32C which are the decoding learning model. The decoders 32Ato 32C have the functions of the segmentation unit 23A, the first reconstruction unit 23B, and the second reconstruction unit 23C, respectively.

The encoder 31 and the latent model 31A as the encoding learning model and the decoders 32A to 32C as the decoding learning model are constructed by performing machine learning using, as training data, a combination of a training image which has the brain including the region of interest as an object and a training label image which corresponds to the region determined according to the type of brain disease in the training image. The encoder 31 and the decoders 32A to 32C consist of, for example, a convolutional neural network (CNN) which is one of multilayer neural networks in which a plurality of processing layers are hierarchically connected. Further, the latent model 31A is trained using a vector quantised-variational auto-encoder (VQ-VAE) method.

The VQ-VAE is a method that is proposed in “Neural Discrete Representation Learning, Aaron van den Oord et al., Advances in Neural Information Processing Systems 30 (NIPS), 6306-6315, 2017” and that receives a latent variable indicating features of input data encoded by a feature amount extractor (that is, an encoder), quantizes the received latent variable, transmits the quantized latent variable to a feature amount decoder (that is, a decoder), and learns the quantization process of the latent variable according to whether or not the original input data has been reconstructed correctly. The learning will be described below.

In addition, the latent model 31A can be trained using any method, such as an auto-encoder method, a variational auto-encoder (VAE) method, a generative adversarial network (GAN) method, or a bidirectional GAN (BiGAN) method, instead of the VQ-VAE.

The convolutional neural network constituting the encoder 31 consists of a plurality of processing layers. Each processing layer is a convolution processing layer and performs a convolution process using various kernels while down-sampling an image input from a processing layer in the previous stage. The kernel has a predetermined pixel size (for example, 3×3), and a weight is set for each element. Specifically, a weight, such as a differential filter that enhances the edge of an input image in the previous stage, is set. Each processing layer applies the kernel to the input image or the entire feature amount output from the processing layer in the previous stage while shifting the pixel of interest of the kernel and outputs a feature map. Further, the processing layer in the later stage in the encoder 31 outputs a feature map with lower resolution. Therefore, the encoder 31 compresses (that is, dimensionally compresses) the features of an input target image G0 such that the resolution of the feature map is reduced to encode the target image G0 and outputs two latent variables, that is, a first latent variable z1 and a second latent variable z2. The first latent variable z1 indicates an image feature for the abnormality of the region of interest in the target image G0, and the second latent variable z2 indicates an image feature for the image in a case in which the region of interest in the target image G0 is a normal region.

Each of the first and second latent variables z1 and z2 consists of n×n D-dimensional vectors. In FIG. 4, for example, n is 4, and the first and second latent variables z1 and z2 can be represented as an n×n map in which each position consists of a D-dimensional vector. In addition, the number of dimensions of the vectors and the number of vectors may be different between the first latent variable z1 and the second latent variable z2. Here, the first latent variable z1 corresponds to a feature vector indicating the image feature for the anomaly of the region of interest. In addition, the second latent variable z2 corresponds to a feature vector indicating the image feature for the image in a case in which the region of interest included in the target image G0 is a normal region.

Here, in this embodiment, in the latent model 31A, K first D-dimensional feature vectors elk indicating a representative image feature for the abnormality of the region of interest are prepared in advance for the first latent variable z1. In addition, in the latent model 31A, K second D-dimensional feature vectors e2k indicating a representative image feature of the image in a case in which the region of interest is a normal region are prepared in advance for the second latent variable z2. In addition, the first feature vectors elk and the second feature vectors e2k are stored in the storage 13. Further, the number of first feature vectors elk prepared and the number of second feature vectors e2k prepared may be different from each other.

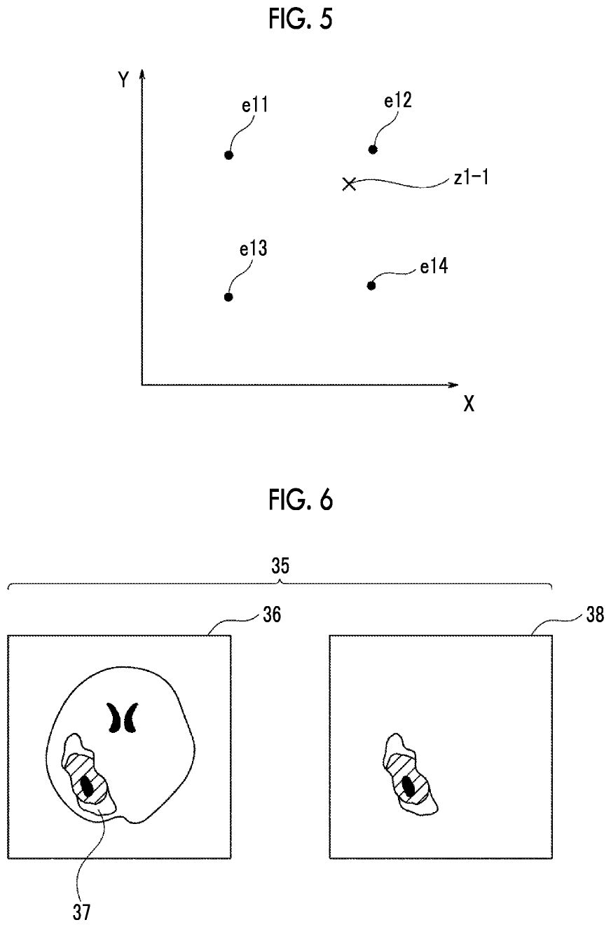

The image encoding device 22 substitutes each of n×n D-dimensional vectors included in the first latent variable z1 with the first feature vector elk in the latent model 31A. In this case, each of the n×n D-dimensional vectors included in the first latent variable z1 is substituted with the first feature vector elk having the minimum difference in a D-dimensional vector space. FIG. 5 is a diagram illustrating the substitution with the first feature vector. In addition, in FIG. 5, for ease of explanation, the vectors of the latent variable are two-dimensionally illustrated. Further, in FIG. 5, it is assumed that four first feature vectors ell to e14 are prepared. As illustrated in FIG. 5, one latent variable vector z1-1 included in the first latent variable z1 has the minimum difference from the first feature vector e12 in the vector space. Therefore, the vector z1-1 is substituted with the first feature vector e12. Further, for the first latent variable z2, similarly to the first latent variable z1, each of n×n D-dimensional vectors is substituted with any one of the second feature vectors e2k.

As described above, the first latent variable z1 is represented by a combination of a maximum of K latent variables having n×n predetermined values by substituting each of the n×n D-dimensional vectors included in the first latent variable z1 with the first feature vector elk. Therefore, first latent variables zd1 are quantized and distributed in a D-dimensional latent space.

Further, the second latent variable z2 is represented by a combination of a maximum of K latent variables having n×n predetermined values by substituting each of the n×n D-dimensional vectors included in the second latent variable z2 with the second feature vector e2k. Therefore, second latent variables zd2 are quantized and distributed in the D-dimensional latent space.

Reference numerals zd1 and zd2 are used as the quantized first and second latent variables. In addition, the quantized first and second latent variables zd1 and zd2 can also be represented as an n×n map in which each position consists of a D-dimensional vector. The quantized first and second latent variables zd1 and zd2 correspond to the first feature amount and the second feature amount, respectively.

The convolutional neural network constituting the decoders 32A to 32C consists of a plurality of processing layers. Each processing layer is a convolution processing layer and performs a convolution process using various kernels while up-sampling the feature amount input from the processing layer in the previous stage in a case in which the first and second latent variables zd1 and zd2 are input as the first and second feature amounts. Each processing layer applies the kernel to the entire feature map consisting of the feature amount output from the processing layer in the previous stage while shifting the pixel of interest of the kernel. Further, the processing layer in the later stage in the decoders 32A to 32C outputs a feature map with higher resolution. In addition, the decoders 32A to 32C do not perform the process in a case in which the similar image search device searches for a similar image as will be described below. However, here, the process performed in the decoders 32A to 32C will be described using the first and second latent variables zd1 and zd2 derived from the target image G0 by the image encoding device 22 since it is required for a learning process which will be described below.

In this embodiment, the first latent variable zd1 is input to the decoder 32A. The decoder 32A derives a region-of-interest label image V0 corresponding to the type of the abnormality of the region of interest in the target image G0 input to the encoder 31 on the basis of the first latent variable zd1.

The second latent variable zd2 is input to the decoder 32B. The decoder 32B derives a first reconstructed image V1 obtained by reconstructing the image feature for the image in a case in which the region of interest included in the target image G0 input to the encoder 31 is a normal region, on the basis of the second latent variable zd2. Therefore, even in a case in which the target image G0 includes the region of interest, the first reconstructed image V1 does not include the region of interest. As a result, the brain included in the first reconstructed image V1 consists of only normal tissue.

The second latent variable zd2 is input to the decoder 32C. In addition, the region-of-interest label image V0 having a size corresponding to the resolution of each processing layer is collaterally input to each processing layer of the decoder 32C. Specifically, a feature map of the region-of-interest label image V0 having a size corresponding to the resolution of each processing layer is collaterally input. In addition, the feature map that is collaterally input may be derived by reducing the feature map output from the processing layer immediately before the region-of-interest label image V0 is derived in the decoder 32A to a size corresponding to the resolution of each processing layer of the decoder 32C. Alternatively, the feature map having the size corresponding to the resolution of each processing layer, which has been derived in the process in which the decoder 32A derives the region-of-interest label image V0, may be input to each processing layer of the decoder 32C. In the following description, it is assumed that the feature map output from the processing layer immediately before the derivation of the region-of-interest label image V0 is reduced to a size corresponding to the resolution of each processing layer of the decoder 32C and then collaterally input to each processing layer of the decoder 32C.

Here, the region-of-interest label image V0 and the feature map are derived on the basis of the first latent variable zd1. Therefore, the decoder 32C derives a second reconstructed image V2 obtained by reconstructing the image feature of the input target image G0 on the basis of the first and second latent variables zd1 and zd2. Therefore, the second reconstructed image V2 is obtained by adding the image feature for the abnormality of the region determined according to the type of disease, which is based on the first latent variable zd1, to the image feature for the brain consisting of only the normal tissues included in the first reconstructed image V1 which is based on the second latent variable zd2. Therefore, the second reconstructed image V2 is obtained by reconstructing the image feature of the input target image G0.

The learning unit 24A of the learning device 24 trains the encoder 31 and the latent model 31A of the image encoding device 22 and the decoders 32A to 32C of the image decoding device 23. FIG. 6 is a diagram illustrating an example of training data used for learning. As illustrated in FIG. 6, training data 35 includes a training image 36 of the brain including a region of interest 37, such as infarction or hemorrhage, and a training label image 38 corresponding to the type of the abnormality of the region of interest in the training image 36.

The learning unit 24A inputs the training image 36 to the encoder 31 and directs the encoder 31 to output the first latent variable z1 and the second latent variable z2 for the training image 36. In addition, in the following description, it is assumed that reference numerals z1 and z2 are also used for the first latent variable and the second latent variable for the training image 36, respectively.

Then, the learning unit 24A substitutes the latent variable vectors included in the first latent variable z1 and in the second latent variable z2 with the first and second feature vectors in the latent model 31A to acquire the quantized first and second latent variables zd1 and zd2. Further, in the following description, it is assumed that reference numerals zd1 and zd2 are also used for the first and second latent variables quantized for the training image 36, respectively. The first and second latent variables zd1 and zd2 quantized for the training image 36 correspond to a first learning feature amount and a second learning feature amount, respectively.

Then, the learning unit 24A inputs the first latent variable zd1 to the decoder 32A to derive a learning region-of-interest label image VT0 corresponding to the type of the abnormality of the region of interest 37 included in the training image 36. In addition, the learning unit 24A inputs the second latent variable zd2 to the decoder 32B to derive a first learning reconstructed image VT1 obtained by reconstructing the image feature for the image in a case in which the region of interest 37 included in the training image 36 is a normal region. Further, the learning unit 24A inputs the second latent variable zd2 to the decoder 32C, collaterally inputs the learning region-of-interest label image VT0 having a size corresponding to the resolution of each processing layer, specifically, the feature map of the learning region-of-interest label image VT0, to each processing layer of the decoder 32C, and derives a second learning reconstructed image VT2 obtained by reconstructing the image feature for the training image 36. In addition, in a case in which second learning reconstructed image VT2 is derived, the feature map output from the processing layer immediately before the learning region-of-interest label image VT0 is derived may be reduced to a size corresponding to the resolution of each processing layer of the decoder 32C and then collaterally input to each processing layer of the decoder 32C.

The learning unit 24A derives a difference between the first latent variable zd1, which is the first learning feature amount, and a predetermined probability distribution of the first feature amount as a first loss L1. Here, the predetermined probability distribution of the first feature amount is a probability distribution that the first latent variable zd1 needs to follow. In a case in which the VQ-VAE method is used, a code word loss and a commitment loss are derived as the first loss L1. The code word loss is a value to be taken by a code word which is a representative local feature in the probability distribution of the first feature amount. The commitment loss is a distance between the first latent variable zd1 and a code word closest to the first latent variable zd1. The encoder 31 and the latent model 31A are trained such that the first latent variable zd1 corresponding to a predetermined probability distribution of the first feature amount is acquired by the first loss L1.

In addition, the learning unit 24A derives a difference between the second latent variable zd2, which is the second learning feature amount, and a predetermined probability distribution of the second feature amount as a second loss L2. Here, the predetermined probability distribution of the second feature amount is a probability distribution that the second latent variable zd2 needs to follow. In a case in which the VQ-VAE method is used, a code word loss and a commitment loss are derived as the second loss L2, similarly to the first loss L1. The code word loss for the second latent variable zd2 is a value to be taken by a code word which is a representative local feature in the probability distribution of the second feature amount. The commitment loss for the second latent variable zd2 is a distance between the second latent variable zd2 and a code word closest to the second latent variable zd2. The encoder 31 and the latent model 31A are trained such that the second latent variable zd2 corresponding to a predetermined probability distribution of the second feature amount is acquired by the second loss L2.

In addition, the learning unit 24A derives, as a third loss L3, the difference between the training label image 38 corresponding to the type of the abnormality of the region of interest 37 included in the training image 36 and in the learning region-of-interest label image VT0 as semantic segmentation for the training image.

The “difference as semantic segmentation” is an index that is determined on the basis of the overlap between a region corresponding to the type of abnormality represented by the training label image 38 and a region corresponding to the type of abnormality represented by the learning region-of-interest label image VT0. Specifically, a value of the number of elements, which are common to the training label image 38 and the learning region-of-interest label image VT0, ×2 for the sum of the number of elements of the training label image 38 and the number of elements of the learning region-of-interest label image VT0 can be used as the difference as semantic segmentation, that is, the third loss L3.

In addition, the learning unit 24A derives the difference between a region other than the region of interest 37 included in the training image 36 and in the first learning reconstructed image VT1 as a fourth loss L4. Specifically, the learning unit 24A derives the difference between a region obtained by removing the region of interest 37 from the training image 36 and from the first learning reconstructed image VT1 as the fourth loss L4.

Further, the learning unit 24A derives the difference between the training image 36 and the second learning reconstructed image VT2 as a fifth loss L5.

Furthermore, the learning unit 24A derives a sixth loss L6 based on the difference between regions corresponding to the inside and outside of the region of interest in the first learning reconstructed image VT1 and in the second learning reconstructed image VT2.

For the sixth loss L6, the first learning reconstructed image VT1 is an image in a case in which the region of interest 37 in the training image 36 is a normal region and is derived not to include the region of interest. On the other hand, the second learning reconstructed image VT2 is derived to include the region of interest. Therefore, in a case in which a difference value between the corresponding pixels of the first learning reconstructed image VT1 and the second learning reconstructed image VT2 is derived, the difference value should be present only in the region corresponding to the region of interest and should not be present in the region that does not correspond to the region of interest. However, in a stage in which the learning has not been ended, the difference value may not be present in the region corresponding to the region of interest since the accuracy of encoding and decoding is low. In addition, the difference value may be present in the region that does not correspond to the region of interest. The sixth loss L6 based on the difference between the regions corresponding to the inside and outside of the region of interest in the first learning reconstructed image VT1 and in the second learning reconstructed image VT2 is an index indicating that, in a case in which the difference value between the corresponding pixels of the first learning reconstructed image VT1 and the second learning reconstructed image VT2 is derived, the difference value is present in the region corresponding to the region of interest and is not present in the region that does not correspond to the region of interest.

Here, as the first latent variable zd1 acquired by the encoder 31 and by the latent model 31A more closely follows a predetermined probability distribution of the first feature amount, the encoder 31 can output the more preferable first latent variable z1 that can faithfully reproduce the abnormality of the region of interest 37 included in the training image 36. In addition, the more preferably quantized first latent variable zd1 can be acquired by the latent model 31A.

Further, as the second latent variable zd2 acquired by the encoder 31 and by the latent model 31A more closely follows a predetermined probability distribution of the second feature amount, the encoder 31 can output the more preferable second latent variable z2 that can faithfully reproduce the image in a case in which the region of interest 37 included in the training image 36 is a normal region. In addition, the more preferably quantized second latent variable zd2 can be acquired by the latent model 31A.

Further, since the learning region-of-interest label image VT0 output from the decoder 32A is derived on the basis of the first latent variable zd1, the learning region-of-interest label image VT0 is not completely matched with the training label image 38. Furthermore, the learning region-of-interest label image VT0 is not completely matched with the region of interest 37 included in the training image 36. However, as the difference between the learning region-of-interest label image VT0 and the training label image 38 as semantic segmentation for the training image 36 becomes smaller, the encoder 31 can output the more preferable first latent variable z1 in a case in which the target image G0 is input. That is, it is possible to output the first latent variable z1 that potentially includes information indicating where the region of interest is in the target image G0 and the image feature for the abnormality of the region of interest. In addition, the more preferably quantized first latent variable zd1 can be acquired by the latent model 31A. Therefore, the first latent variable zd1 indicating the image feature for the abnormality of the region of interest is derived while the region of interest is being extracted from the target image G0 by the encoder 31. In addition, the decoder 32A can output the region-of-interest label image V0 corresponding to the type of the abnormality of the region of interest, for the region corresponding to the region of interest included in the target image.

Further, since the first learning reconstructed image VT1 output from the decoder 32B is derived on the basis of the second latent variable zd2, the first learning reconstructed image VT1 is not completely matched with the image feature for the image in a case in which the region of interest 37 included in the training image 36 is a normal region. However, as the difference between the first learning reconstructed image VT1 and a region other than the region of interest 37 in the training image 36 becomes smaller, the encoder 31 can output the more preferable second latent variable z2 in a case in which the target image G0 is input. In addition, the more preferably quantized second latent variable zd2 can be acquired by the latent model 31A. Further, the decoder 32B can output the first reconstructed image V1 that is closer to the image feature for the image in a case in which the region of interest included in the target image G0 is a normal region.

Furthermore, since the second learning reconstructed image VT2 output from the decoder 32C is derived on the basis of the first latent variable zd1 and the second latent variable zd2, the second learning reconstructed image VT2 is not completely matched with the training image 36. However, as the difference between the second learning reconstructed image VT2 and the training image 36 becomes smaller, the encoder 31 can output the more preferable first and second latent variables z1 and z2 in a case in which the target image G0 is input. In addition, the more preferably quantized first latent variable zd1 and second latent variable zd2 can be acquired by the latent model 31A. Further, the decoder 32C can output the second reconstructed image V2 that is closer to the target image G0.

Furthermore, there is a difference in the presence or absence of the region of interest between the first learning reconstructed image VT1 output from the decoder 32B and the second learning reconstructed image VT2 output from the decoder 32C. Therefore, the more the difference value between the first learning reconstructed image VT1 and the second learning reconstructed image VT2 is secured to be equal to or greater than a certain value in a region corresponding to the region of interest and the smaller the absolute value of the difference between the first learning reconstructed image VT1 and the second learning reconstructed image VT2 becomes in a region that does not correspond to the region of interest, the more preferable first and second latent variables z1 and z2 can be output by the encoder 31 in a case in which the target image G0 is input. In addition, the more preferably quantized first latent variable zd1 and second latent variable zd2 can be acquired by the latent model 31A. Further, the decoder 32B can output the first reconstructed image V1 that is closer to the image in a case in which the region of interest included in the target image G0 is a normal region. Furthermore, the decoder 32C can output the second reconstructed image V2 that is closer to the target image G0.

Therefore, the learning unit 24A trains the encoder 31, the latent model 31A, and the decoders 32A to 32C on the basis of at least one of the first to sixth losses L1 to L6 derived as described above. In this embodiment, the learning unit 24A trains the encoder 31, the latent model 31A, and the decoders 32A to 32C such that all of the first to sixth losses L1 to L6 satisfy predetermined conditions. That is, the encoder 31 and the decoders 32A to 32C are trained by deriving, for example, the number of processing layers and the number of pooling layers constituting the encoder 31 and the decoders 32A to 32C, coefficients of the kernels in the processing layers, the size of the kernels, and weights for the connections between the layers such that the first to fifth losses L1 to L5 are reduced and the sixth loss L6 has an appropriate value. Further, the learning unit 24A updates the first feature vector elk and the second feature vector e2k for the latent model 31A such that the first to fifth losses L1 to L5 are reduced and the sixth loss L6 has an appropriate value.

In addition, in this embodiment, the learning unit 24A trains the encoder 31, the latent model 31A, and the decoders 32A to 32C such that the first loss L1 is equal to or less than a predetermined threshold value Th1, the second loss L2 is equal to or less than a predetermined threshold value Th2, the third loss L3 is equal to or less than a predetermined threshold value Th3, the fourth loss L4 is equal to or less than a predetermined threshold value Th4, and the fifth loss L5 is equal to or less than a predetermined threshold value Th5. Further, the learning unit 24A trains the encoder 31, the latent model 31A, and the decoders 32A to 32C such that, for the sixth loss L6, the absolute value of the difference between the first learning reconstructed image VT1 and the second learning reconstructed image VT2 is equal to or greater than a predetermined threshold value Th6 in the region corresponding to the region of interest and the difference value between the first learning reconstructed image VT1 and the second learning reconstructed image VT2 is equal to or less than a predetermined threshold value Th7 in the region that does not correspond to the region of interest. In addition, instead of the learning using the threshold value, the learning may be performed a predetermined number of times, or the learning may be performed such that each of the losses L1 to L6 is the minimum value or the maximum value.

In a case in which the learning unit 24A trains the encoder 31, the latent model 31A, and the decoders 32A to 32C in this way, the encoder 31 outputs the first latent variable z1 that more appropriately indicates the image feature for the abnormality of the region of interest of the brain included in the input target image G0. In addition, the encoder 31 outputs the second latent variable z2 that more appropriately indicates the image feature of the brain in a case in which the region of interest is a normal region in the brain included in the input target image G0. In addition, the latent model 31A acquires the quantized first latent variable zd1 that more appropriately indicates the image feature indicating the abnormality of the region of interest of the brain included in the input target image G0. Further, the latent model 31A acquires the quantized second latent variable zd2 that more appropriately indicates the image feature of the brain in a case in which the region of interest is a normal region in the brain included in the input target image G0.

In addition, the decoder 32A outputs the region-of-interest label image V0 which more accurately indicates semantic segmentation corresponding to the type of the abnormality of the region of interest included in the target image G0 in a case in which the quantized first latent variable zd1 is input. Further, in a case in which the quantized second latent variable zd2 is input, the decoder 32B outputs the first reconstructed image V1 obtained by reconstructing the image feature of the brain in a case in which the region of interest in the target image G0 is a normal region. Furthermore, in a case in which the quantized second latent variable zd2 is input and the region-of-interest label image V0 is collaterally input to each processing layer, the decoder 32C adds the image feature for the abnormality of the region determined according to the type of disease based on the first latent variable zd1 to the image feature of the brain consisting of only the normal tissues included in the first reconstructed image V1 based on the second latent variable zd2. As a result, the decoder 32C outputs the second reconstructed image V2 obtained by reconstructing the image feature of the brain including the region of interest.

The similarity derivation unit 25A of the similar image search device 25 derives similarities between the query image (that is, the target image G0) to be diagnosed and all of the reference images registered in the image database DB stored in the image storage server 3 in order to search for a similar reference image that is similar to the query image among the reference images registered in the image database DB. In addition, in the following description, it is assumed that the query image is denoted by the same reference numeral G0 as the target image. Here, a plurality of reference images for various cases of the brain are registered in the image database DB. In this embodiment, for the reference images, the quantized first and second latent variables are derived in advance by the image encoding device 22 including the trained encoder 31 and are registered in the image database DB in association with the reference images. The first and second latent variables registered in the image database DB in association with the reference images are referred to as first and second reference latent variables, respectively.

Hereinafter, the derivation of the similarity by the similarity derivation unit 25A will be described. In this embodiment, it is assumed that the query image G0 includes the region of interest which is a brain disease. The similarity derivation unit 25A derives the similarity between the query image G0 and the reference image on the basis of the search conditions.

Here, in this embodiment, the image encoding device 22 derives the first latent variable indicating the image feature for the abnormality of the region of interest included in the query image G0. In addition, the image encoding device 22 derives the second latent variable indicating the image feature for the image in a case in which the region of interest in the query image G0 is a normal region. Therefore, in this embodiment, it is possible to select, as the search conditions, a first search condition for searching for a reference image that is similar to the query image G0 including the region of interest, a second search condition for searching for a reference image that is similar only in the abnormality of the region of interest included in the query image G0, and a third search condition for searching for a reference image that is similar to the image in a case in which the region of interest included in the query image G0 is a normal region. The selection can be input to the image processing system 20 by the input device 15. Then, the similarity derivation unit 25A derives the similarity between the query image G0 and the reference image according to the input search condition.

In a case in which the first search condition is input, the similarity derivation unit 25A derives the similarity on the basis of the difference between the first latent variable zd1 derived for the query image G0 and the first reference latent variable corresponding to the reference image and the difference between the second latent variable zd2 derived for the query image G0 and the second reference latent variable corresponding to the reference image.

Specifically, as illustrated in the following Expression (1), the similarity derivation unit 25A derives a Euclidean distance √{(Vt1(i, j)−Vr1(i, j)}² between the corresponding position vectors of the first latent variable zd1 and the first reference latent variable in the map in the vector space of the latent variable and derives the sum of the derived Euclidean distances Σ[√{(Vt1(i, j)−Vr1(i, j)}²]. In addition, the similarity derivation unit 25A derives a Euclidean distance √{(Vt2(i, j)−Vr2(i, j)}² between the corresponding position vectors of the second latent variable zd2 and the second reference latent variable in the map and derives the sum of the derived Euclidean distances Σ[√{(Vt2(i, j)−Vr2(i, j)}²]. Then, the similarity derivation unit 25A derives the sum of the two sums as the similarity.

In Expression (1), S1 indicates the similarity based on the first search condition, Vt1(i, j) indicates a vector at a map position (i, j) in the first latent variable zd1, Vr1(i, j) indicates a vector at a map position (i, j) in the first reference latent variable, Vt2(i, j) indicates a vector at a map position (i, j) in the second latent variable zd2, and Vr2(i, j) indicates a vector at a map position (i, j) in the second reference latent variable.

S1=Σ[√{(Vt1(i, j)−Vr1(i, j)}²]+Σ[√{(Vt2(i, j)−Vr2(i, j)}²] (1)

In addition, the similarity S1 may be derived by the following Expression (1a) instead of the above-described Expression (1). Here, concat(a, b) is an operation of connecting a vector a and a vector b.

S1=Σ[√{(Vt12(i, j)−Vr12(i, j)}²] (1a)

where

Vt12(i, j)=concat(Vt1(i, j), Vt2(i, j))

Vr12(i, j)=concat(Vr1(i, j), Vr2(i, j))

On the other hand, in a case in which the second search condition is input, the similarity derivation unit 25A derives the similarity on the basis of the difference between the first latent variable zd1 derived for the query image G0 and the first reference latent variable corresponding to the reference image. Specifically, as illustrated in the following Expression (2), the similarity derivation unit 25A derives the Euclidean distance √{(Vt1(i, j)−Vr1(i, j)}² between the corresponding position vectors of the first latent variable zd1 and the first reference latent variable in the map in the vector space of the latent variable and derives the sum of the derived Euclidean distances Σ[√{(Vt1(i, j)−Vr1(i, j)}²] as a similarity S2.

S2=Σ[√{(Vt1(i, j)−Vr1(i, j)}²] (2)

Further, in a case in which the third search condition is input, the similarity derivation unit 25A derives the similarity on the basis of the difference between the second latent variable zd2 derived for the query image G0 and the second reference latent variable corresponding to the reference image. Specifically, as illustrated in the following Expression (3), the similarity derivation unit 25A derives the Euclidean distance √{(Vt2(i, j)−Vr2(i, j)}² between the corresponding position vectors of the second latent variable zd2 and the second reference latent variable in the map in the vector space of the latent variable and derives the sum of the derived Euclidean distances Σ[√{(Vt2(i, j)−Vr2(i, j)}²] as a similarity S3.

S3=[√{(Vt2(i, j)−Vr2(i, j)}²] (3)

The derivation of the similarities S1 to S3 is not limited to the above-described method. For example, a Manhattan distance, a vector inner product, or a cosine similarity may be used instead of the Euclidean distance.

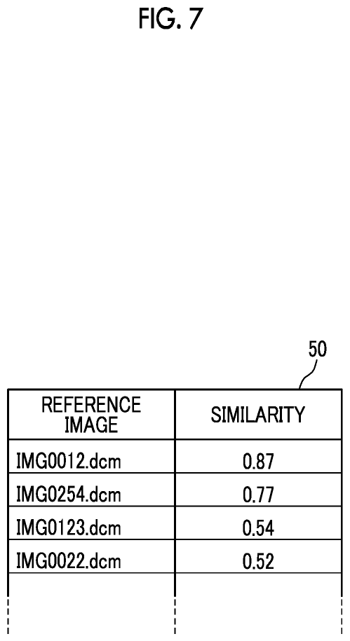

The extraction unit 25B of the similar image search device 25 extracts a similar reference image that is similar to the query image G0 from the image database DB on the basis of the similarities S1 to S3 corresponding to the input search conditions. The extraction unit 25B extracts a reference image that is similar to the target image G0 as the similar reference image on the basis of the similarities S1 to S3 between the query image G0 and all of the reference images registered in the image database DB. Specifically, the extraction unit 25B sorts the reference images in descending order of the similarities S1 to S3 and creates a search result list. FIG. 7 is a diagram illustrating the search result list. As illustrated in FIG. 7, the reference images registered in the image database DB are sorted in descending order of the similarities S1 to S3 in a search result list 50. Then, the extraction unit 25B extracts a predetermined number of reference images sorted in descending order of the similarity in the search result list 50 as the similar reference images from the image database DB.

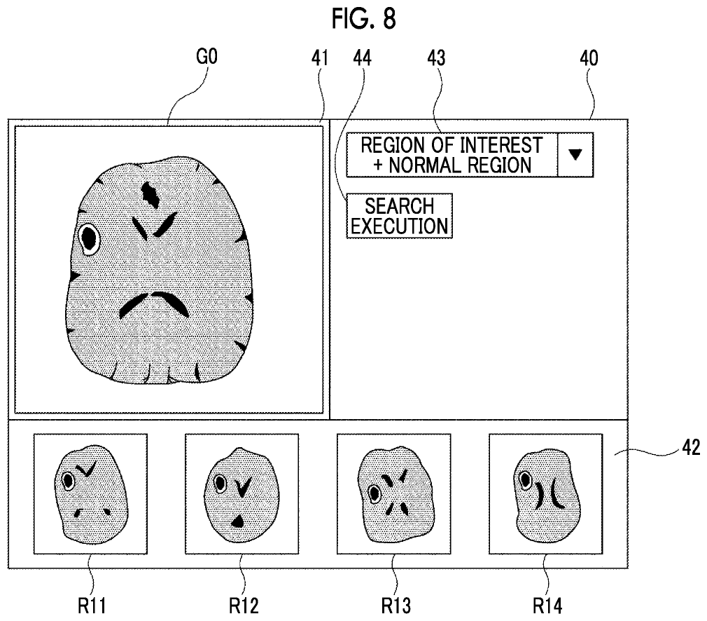

The display control unit 26 displays the extraction results by the extraction unit 25B on the display 14. FIGS. 8 to 10 are diagrams illustrating display screens for the extraction results based on the first to third search conditions. As illustrated in FIGS. 8 to 10, a display screen 40 for the extraction results includes a first display region 41 in which the query image G0 is displayed and a second display region 42 in which the search results are displayed. In addition, the display screen 40 includes a pull-down menu 43 for selecting the search condition and a search execution button 44 for executing the search. Further, the pull-down menu 43 can be used to select “the region of interest +the normal region” indicating the first search condition, “only the region of interest” indicating the second search condition, and “only the normal region” indicating the third search condition. The operator selects a desired search condition in the pull-down menu 43 and selects the search execution button 44. Then, the process according to this embodiment is executed, and the display screen 40 for the search results is displayed on the display 14.

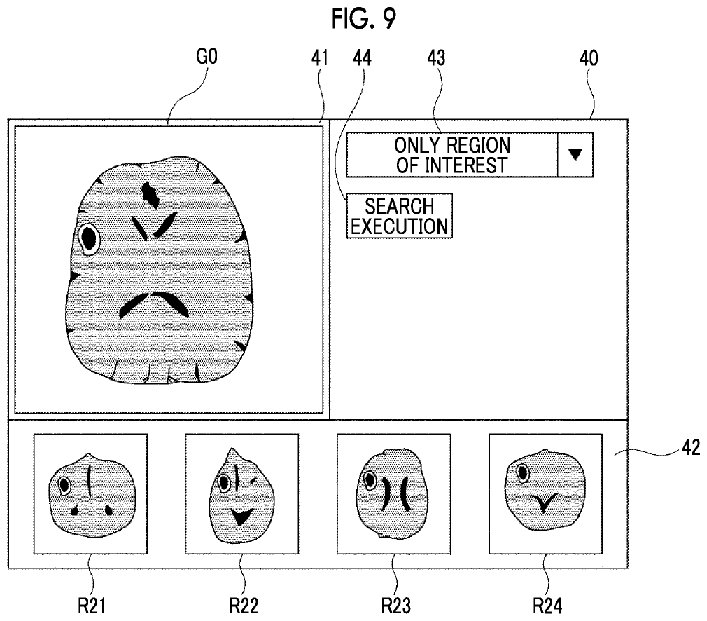

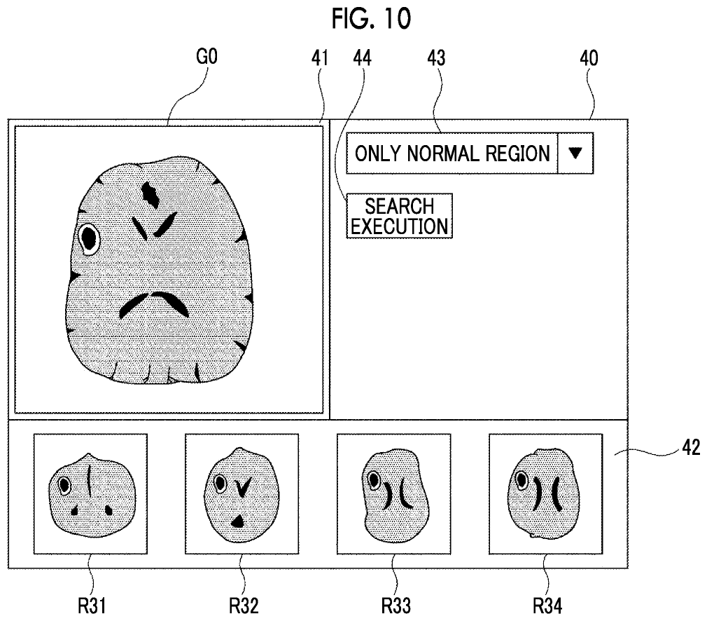

As illustrated in FIG. 8, four similar reference images R11 to R14 which include the region of interest included in the query image G0 and which are similar to the query image G0 are displayed in the second display region 42 of the display screen 40 for the search results based on the first search condition. In addition, as illustrated in FIG. 9, four similar reference images R21 to R24 which are similar only in the abnormality of the region of interest included in the query image G0 are displayed in the second display region 42 of the display screen based on the second search condition. In addition, as illustrated in FIG. 10, four similar reference images R31 to R34 which are similar to the image in a case in which the region of interest is a normal region in the brain included in the query image G0 are displayed in the second display region 42 of the display screen 40 for the search results based on the third search condition.

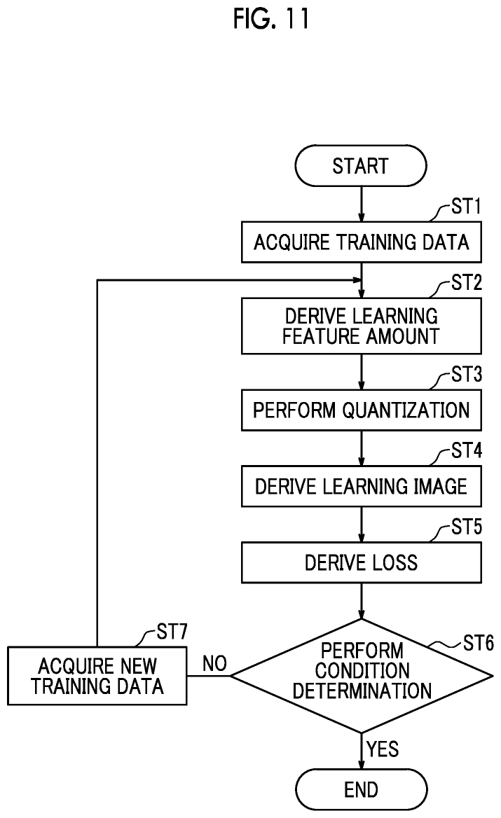

Next, a process performed in this embodiment will be described. FIG. 11 is a flowchart illustrating a learning process performed in this embodiment. In addition, it is assumed that a plurality of training data items are acquired from the image storage server 3 and are stored in the storage 13. First, the learning unit 24A of the learning device 24 acquires one training data item 35 including the training image 36 and the training label image 38 from the storage 13 (Step ST1) and inputs the training image 36 included in the training data 35 to the encoder 31 of the image encoding device 22. The encoder 31 derives the first latent variable z1 and the second latent variable z2 as the first learning feature amount and the second learning feature amount, respectively (learning feature amount derivation; Step ST2).

Then, the learning unit 24A derives the quantized first latent variable zd1 and the quantized second latent variable zd2 from the first latent variable z1 and the second latent variable z2, respectively (quantization; Step ST3). Then, the learning unit 24A inputs the quantized first latent variable zd1 to the decoder 32A of the image decoding device 23. Then, the decoder 32A derives the learning region-of-interest label image VT0 corresponding to the type of the abnormality of the region of interest 37 from the training image 36. In addition, the learning unit 24A inputs the quantized second latent variable zd2 to the decoder 32B of the image decoding device 23. Then, the decoder 32B derives the first learning reconstructed image VT1 obtained by reconstructing the image in a case in which the region of interest included in the training image 36 is a normal region. Further, the learning unit 24A inputs the second latent variable zd2 to the decoder 32C and collaterally inputs the learning region-of-interest label image VT0 having a size corresponding to the resolution of each processing layer of the decoder 32C to each processing layer of the decoder 32C. Then, the decoder 32C derives the second learning reconstructed image VT2 obtained by reconstructing the image feature of the training image 36 (learning image derivation; Step ST4).

Then, the learning unit 24A derives the first to sixth losses L1 to L6 as described above (Step ST5).

Then, the learning unit 24A determines whether or not the first to sixth losses L1 to L6 satisfy predetermined conditions (condition determination; Step ST6). In a case in which the determination result in Step ST6 is “No”, the learning unit 24A acquires new training data from the storage 13 (Step ST7), returns to the process in Step ST2, and repeats the processes in Steps ST2 to ST6 using the new training data. In a case in which the determination result in Step ST6 is “Yes”, the learning unit 24A ends the learning process. As a result, the encoder 31 of the image encoding device 22 and the decoders 32A to 32C of the image decoding device 23 are constructed.

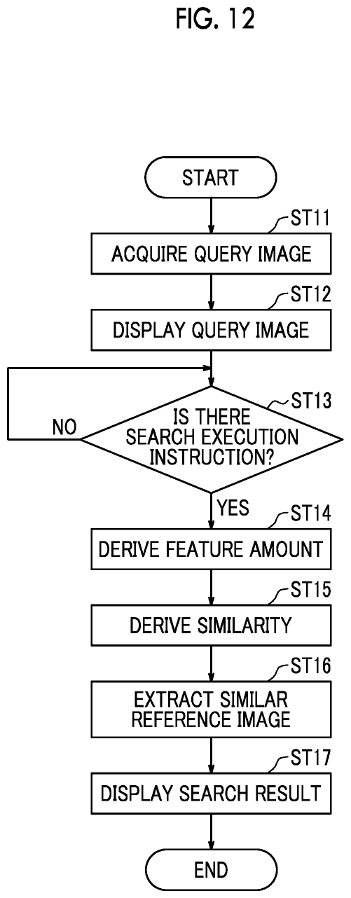

Next, a similar image search process performed in this embodiment will be described. FIG. 12 is a flowchart illustrating the similar image search process performed in this embodiment. First, the information acquisition unit 21 acquires the query image G0 to be searched for (Step ST11), and the display control unit 26 displays the query image G0 on the display 14 (Step ST12). Then, in a case in which the search condition is specified in the pull-down menu 43 and the search execution button 44 is selected to send instruction for search execution (Step ST13; YES), the image encoding device 22 derives the quantized first latent variable zd1 and the quantized second latent variable zd2 for the query image G0 as the first feature amount and the second feature amount, respectively (feature amount derivation; Step ST14). Then, the similarity derivation unit 25A derives the similarities between the target image G0 and the reference images registered in the image database DB of the image storage server 3 on the basis of the first and second feature amounts (Step ST15). Then, the extraction unit 25B extracts a predetermined number of reference images having the highest similarity as the similar reference images according to the search condition (Step ST16). Further, the display control unit 26 displays the similar reference images in the second display region 42 of the display screen 40 (search result display; Step ST17). Then, the process ends.

As described above, in this embodiment, the encoder 31 of the image encoding device 22 encodes the target image G0 to derive at least one first feature amount indicating the image feature for the abnormality of the region of interest included in the target image G0. In addition, the encoder 31 encodes the target image G0 to derive at least one second feature amount indicating the image feature for the image in a case in which the region of interest included in the target image G0 is a normal region. Therefore, the encoding of the target image G0 makes it possible to separately treat the image feature for the abnormality of the region of interest included in the target image G0 and the image feature for the image in a case in which the region of interest is a normal region.

In addition, the image feature for the region determined according to the type of the disease included in the region of interest included in the target image G0 is treated as the difference from the image feature for the image in a case in which the region of interest is a normal region, which makes it possible to search for a reference image that is similar to the target image G0 using only the first feature amount indicating the image feature for the abnormality of the region of interest included in the target image G0. In addition, it is possible to search for a reference image that is similar to the target image G0 using only the second feature amount indicating the image feature of the image in a case in which the region of interest included in the target image G0 is a normal region. Further, it is possible to search for a reference image that is similar to the target image G0 using both the first and second feature amounts. Therefore, it is possible to search for a similar image corresponding to a desired search condition.

Furthermore, in this embodiment, the trained decoder 32A of the image decoding device 23 can be used to derive the region-of-interest label image V0 corresponding to the type of the abnormality of the region of interest included in the input target image G0 from the first feature amount. Therefore, it is possible to acquire, as a label image, a region determined according to the type of the disease included in the target image G0.

In addition, in this embodiment, the trained decoder 32B of the image decoding device 23 can be used to derive the first reconstructed image V1 obtained by reconstructing the image feature for the image in a case in which the region of interest included in the input target image G0 is a normal region from the second feature amount. Therefore, it is possible to acquire an image that consists of only the normal tissues obtained by removing the region of interest from the input image.

Further, in this embodiment, the trained decoder 32C of the image decoding device 23 can be used to derive the second reconstructed image V2 obtained by reconstructing the image feature for the target image G0. Therefore, it is possible to reproduce the target image G0.

Furthermore, in the image encoding device according to this embodiment, in a case in which the target image does not include an abnormal region as the region of interest, the first feature amount is an invalid value. In this case, the second feature amount or a combination of the first feature amount and the second feature amount may indicate the image feature for the target image.

In addition, in the above-described embodiment, the image of the brain is used as the target image. However, the target image is not limited to the image of the brain. An image including any part of the human body, such as a lung, a heart, a liver, a kidney, and limbs, in addition to the brain can be used as the target image. In this case, the encoder 31 and the decoders 32A to 32C may be trained using the training image and the training label image including diseases, such as a tumor, an infarction, a cancer, and a bone fracture, appearing in the part as the region of interest. Therefore, it is possible to derive, from the target image G0, the first feature amount indicating the image feature for the abnormality of the region of interest corresponding to the part included in the target image G0 and the second feature amount indicating the image feature for the image in a case in which the region of interest included in the target image G0 is a normal region.

In addition, in the above-described embodiment, separate encoding learning models may be used for the first feature amount derivation unit 22A and the second feature amount derivation unit 22B, and the first feature amount and the second feature amount may be derived by the separate encoding learning models.

Further, in the above-described embodiment, for example, the following various processors can be used as a hardware structure of processing units performing various processes, such as the information acquisition unit 21, the first feature amount derivation unit 22A, the second feature amount derivation unit 22B, the segmentation unit 23A, the first reconstruction unit 23B, the second reconstruction unit 23C, the learning unit 24A, the similarity derivation unit 25A, the extraction unit 25B, and the display control unit 26. The various processors include, for example, a CPU which is a general-purpose processor executing software (programs) to function as various processing units as described above, a programmable logic device (PLD), such as a field programmable gate array (FPGA), which is a processor whose circuit configuration can be changed after manufacture, and a dedicated electric circuit, such as an application specific integrated circuit (ASIC), which is a processor having a dedicated circuit configuration designed to perform a specific process.

One processing unit may be configured by one of the various processors or by a combination of two or more processors of the same type or different types (for example, a combination of a plurality of FPGAs or a combination of a CPU and an FPGA). In addition, a plurality of processing units may be configured by one processor.

A first example of the configuration in which a plurality of processing units are configured by one processor is an aspect in which one processor is configured by a combination of one or more CPUs and software and functions as a plurality of processing units. A representative example of this aspect is a client computer or a server computer. A second example of the configuration is an aspect in which a processor that implements the functions of the entire system including a plurality of processing units using one integrated circuit (IC) chip is used. A representative example of this aspect is a system-on-chip (SoC). As described above, various processing units are configured by one or more of the various processors as a hardware structure.

In addition, specifically, an electric circuit (circuitry) obtained by combining circuit elements, such as semiconductor elements, can be used as the hardware structure of the various processors.

What is claimed is:

1. An image encoding device comprising:
at least one processor,
wherein the processor is configured to encode a target image to derive at least one first feature amount indicating an image feature for an abnormality of a region of interest included in the target image and to encode the target image to derive at least one second feature amount indicating an image feature for an image in a case in which the region of interest included in the target image is a normal region.

2. The image encoding device according to claim 1,
wherein a combination of the first feature amount and the second feature amount indicates an image feature for the target image.

3. The image encoding device according to claim 1, further comprising:
a storage that stores at least one first feature vector indicating a representative image feature for the abnormality of the region of interest and at least one second feature vector indicating a representative image feature for the image in a case in which the region of interest is the normal region,
wherein the processor is configured to derive the first feature amount by substituting a feature vector indicating the image feature for the abnormality of the region of interest with a first feature vector, which minimizes a difference from the image feature for the abnormality of the region of interest, among the first feature vectors to quantize the feature vector and to derive the second feature amount by substituting a feature vector indicating the image feature for the image in a case in which the region of interest is the normal region with a second feature vector, which minimizes a difference from the image feature for the image in a case in which the region of interest is the normal region, among the second feature vectors to quantize the feature vector.

4. The image encoding device according to claim 1,
wherein the processor is configured to derive the first feature amount and the second feature amount, using an encoding learning model which has been trained to derive the first feature amount and the second feature amount in a case in which the target image is input.

5. An image decoding device comprising:
at least one processor,
wherein the processor is configured to extract a region corresponding to a type of the abnormality of the region of interest in the target image on the basis of the first feature amount derived from the target image by the image encoding device according to claim 1.

6. The image decoding device according to claim 5,
wherein the processor is configured to derive a first reconstructed image obtained by reconstructing an image feature for an image in a case in which the region of interest in the target image is a normal region on the basis of the second feature amount and to derive a second reconstructed image obtained by reconstructing an image feature for the target image on the basis of the first feature amount and the second feature amount.

7. The image decoding device according to claim 6,
wherein the processor is configured to derive a label image corresponding to the type of the abnormality of the region of interest in the target image, the first reconstructed image, and the second reconstructed image, using a decoding learning model which has been trained to derive the label image corresponding to the type of the abnormality of the region of interest in the target image on the basis of the first feature amount, to derive the first reconstructed image obtained by reconstructing the image feature for the image in a case in which the region of interest in the target image is the normal region on the basis of the second feature amount, and to derive the second reconstructed image obtained by reconstructing the image feature of the target image on the basis of the first feature amount and the second feature amount.

8. An image processing device comprising:
the image encoding device according to claim 1; and
the image decoding device according to claim 5.

9. A learning device that trains the encoding learning model in the image encoding device according to claim 4 and the decoding learning model in the image decoding device according to claim 7, using training data consisting of a training image including a region of interest and a training label image corresponding to a type of an abnormality of the region of interest in the training image, the learning device comprising:
at least one processor,
wherein the processor is configured to derive a first learning feature amount and a second learning feature amount corresponding to the first feature amount and the second feature amount, respectively, from the training image using the encoding learning model, to derive a learning label image corresponding to the type of the abnormality of the region of interest included in the training image on the basis of the first learning feature amount, to derive a first learning reconstructed image obtained by reconstructing an image feature for an image in a case in which the region of interest in the training image is a normal region on the basis of the second learning feature amount, and to derive a second learning reconstructed image obtained by reconstructing an image feature for the training image on the basis of the first learning feature amount and the second learning feature amount, using the decoding learning model, and to train the encoding learning model and the decoding learning model such that at least one of a first loss which is a difference between the first learning feature amount and a predetermined probability distribution of the first feature amount, a second loss which is a difference between the second learning feature amount and a predetermined probability distribution of the second feature amount, a third loss based on a difference between the training label image included in the training data and the learning label image as semantic segmentation for the training image, a fourth loss based on a difference between the first learning reconstructed image and an image outside the region of interest in the training image, a fifth loss based on a difference between the second learning reconstructed image and the training image, or a sixth loss based on a difference between regions corresponding to an inside and an outside of the region of interest in the first learning reconstructed image and in the second learning reconstructed image satisfies a predetermined condition.

10. A similar image search device comprising:
at least one processor; and
the image encoding device according to claim 1,
wherein the processor is configured to derive a first feature amount and a second feature amount for a query image using the image encoding device, to derive a similarity between the query image and each of a plurality of reference images on the basis of at least one of the first feature amount or the second feature amount derived from the query image with reference to an image database in which a first feature amount and a second feature amount for each of the plurality of reference images are registered in association with each of the plurality of reference images, and to extract a reference image that is similar to the query image as a similar image from the image database on the basis of the similarity.

11. An image encoding method comprising:
encoding a target image to derive at least one first feature amount indicating an image feature for an abnormality of a region of interest included in the target image; and
encoding the target image to derive at least one second feature amount indicating an image feature for an image in a case in which the region of interest included in the target image is a normal region.

12. An image decoding method comprising:
extracting a region corresponding to a type of an abnormality of the region of interest in the target image on the basis of the first feature amount derived from the target image by the image encoding device according to claim 1.

13. A learning method for training the encoding learning model in the image encoding device according to claim 4 and the decoding learning model in the image decoding device according to claim 7, using training data consisting of a training image including a region of interest and a training label image corresponding to a type of an abnormality of the region of interest in the training image, the learning method comprising:
deriving a first learning feature amount and a second learning feature amount corresponding to the first feature amount and the second feature amount, respectively, from the training image using the encoding learning model;
deriving a learning label image corresponding to the type of the abnormality of the region of interest included in the training image on the basis of the first learning feature amount, deriving a first learning reconstructed image obtained by reconstructing an image feature for an image in a case in which the region of interest in the training image is a normal region on the basis of the second learning feature amount, and deriving a second learning reconstructed image obtained by reconstructing an image feature for the training image on the basis of the first learning feature amount and the second learning feature amount, using the decoding learning model; and
training the encoding learning model and the decoding learning model such that at least one of a first loss which is a difference between the first learning feature amount and a predetermined probability distribution of the first feature amount, a second loss which is a difference between the second learning feature amount and a predetermined probability distribution of the second feature amount, a third loss based on a difference between the training label image included in the training data and the learning label image as semantic segmentation for the training image, a fourth loss based on a difference between the first learning reconstructed image and an image outside the region of interest in the training image, a fifth loss based on a difference between the second learning reconstructed image and the training image, or a sixth loss based on a difference between regions corresponding to an inside and an outside of the region of interest in the first learning reconstructed image and in the second learning reconstructed image satisfies a predetermined condition.

14. A similar image search method comprising:
deriving a first feature amount and a second feature amount for a query image using the image encoding device according to claim 1;
deriving a similarity between the query image and each of a plurality of reference images on the basis of at least one of the first feature amount or the second feature amount derived from the query image with reference to an image database in which a first feature amount and a second feature amount for each of the plurality of reference images are registered in association with each of the plurality of reference images; and
extracting a reference image that is similar to the query image as a similar image from the image database on the basis of the similarity.

15. A non-transitory computer-readable storage medium that stores an image encoding program that causes a computer to execute:
a procedure of encoding a target image to derive at least one first feature amount indicating an image feature for an abnormality of a region of interest included in the target image; and
a procedure of encoding the target image to derive at least one second feature amount indicating an image feature for an image in a case in which the region of interest included in the target image is a normal region.

16. A non-transitory computer-readable storage medium that stores an image decoding program that causes a computer to execute:
a procedure of extracting a region corresponding to a type of an abnormality of the region of interest in the target image on the basis of the first feature amount derived from the target image by the image encoding device according to claim 1.

17. A non-transitory computer-readable storage medium that stores a learning program that causes a computer to execute a procedure of training the encoding learning model in the image encoding device according to claim 4 and the decoding learning model in the image decoding device according to claim 7, using training data consisting of a training image including a region of interest and a training label image corresponding to a type of an abnormality of the region of interest in the training image, the learning program causing the computer to execute:
a procedure of deriving a first learning feature amount and a second learning feature amount corresponding to the first feature amount and the second feature amount, respectively, from the training image using the encoding learning model;
a procedure of deriving a learning label image corresponding to the type of the abnormality of the region of interest included in the training image on the basis of the first learning feature amount, deriving a first learning reconstructed image obtained by reconstructing an image feature for an image in a case in which the region of interest in the training image is a normal region on the basis of the second learning feature amount, and deriving a second learning reconstructed image obtained by reconstructing an image feature for the training image on the basis of the first learning feature amount and the second learning feature amount, using the decoding learning model; and
a procedure of training the encoding learning model and the decoding learning model such that at least one of a first loss which is a difference between the first learning feature amount and a predetermined probability distribution of the first feature amount, a second loss which is a difference between the second learning feature amount and a predetermined probability distribution of the second feature amount, a third loss based on a difference between the training label image included in the training data and the learning label image as semantic segmentation for the training image, a fourth loss based on a difference between the first learning reconstructed image and an image outside the region of interest in the training image, a fifth loss based on a difference between the second learning reconstructed image and the training image, or a sixth loss based on a difference between regions corresponding to an inside and an outside of the region of interest in the first learning reconstructed image and in the second learning reconstructed image satisfies a predetermined condition.

18. A non-transitory computer-readable storage medium that stores a similar image search program that causes a computer to execute:
a procedure of deriving a first feature amount and a second feature amount for a query image using the image encoding device according to claim 1;
a procedure of deriving a similarity between the query image and each of a plurality of reference images on the basis of at least one of the first feature amount or the second feature amount derived from the query image with reference to an image database in which a first feature amount and a second feature amount for each of the plurality of reference images are registered in association with each of the plurality of reference images; and
a procedure of extracting a reference image that is similar to the query image as a similar image from the image database on the basis of the similarity.
